(19) United States
(12) Patent Application Publication
(10) Pub. No.: US 2020/0147151 A1
(43) Pub. Date: May 14, 2020

(54) COMPOSITIONS AND METHODS FOR FECAL MICROBIOTA TRANSPLANTATION

(71) Applicant: THE BOARD OF REGENTS OF THE UNIVERSITY OF TEXAS SYSTEM, Austin, TX (US)

(72) Inventors: Herbert L. DUPONT, Houston, TX (US); Zhi-Dong JIANG, Houston, TX (US)

(21) Appl. No.: 16/481,187

(22) PCT Filed: Jan. 30, 2018
(86) PCT No.: PCT/US2018/015915

§ 371 Date: Jul. 26, 2019

Related U.S. Application Data
(60) Provisional application No. 62/452,325, filed on Jan. 30, 2017.

(51) Int. Cl. A61K 35/742; A61K 47/26

(52) U.S. Cl. CPC A61K 35/742; A61K 9/0031; A61K 47/26

(57) ABSTRACT

The present disclosure provides human fecal microbiota compositions, including fresh, frozen, and lyophilized compositions, for the treatment of disorders associated with dysfunctional microbiota, such as recurrent Clostridium difficile infection. In particular, the fecal microbiota composition comprises a cryoprotectant sugar, such as mannitol.

This application claims the benefit of U.S. Provisional Patent Application No. 62/452,325, filed Jan. 30, 2017, the entirety of which is incorporated herein by reference.

### BACKGROUND OF THE INVENTION

### 1. Field of the Invention

The present invention relates generally to the fields of microbiology and medicine. More particularly, it concerns compositions of fecal microbiota and uses thereof.

### 2. Description of Related Art

CDI recurrence is associated with reduced diversity of fecal microbiota and impaired microbial community resilience and colonization resistance (1). In a study of elderly hospitalized patients with CDI, gut microbiota showed lower proportions for those taxa previously shown to be associated with diverse healthy intestinal microbiota, Bacteroides, Alistipes, Lachnospira or Barnesiella, when compared with elderly patients receiving antibiotics in the hospital but without CDI (2).

Fecal microbiota transplantation (FMT) has become standard for therapy of recurrent CDI with clinical cure rates ≥80% (3-8) with most centers using frozen fecal products (5, 8-10). Preliminary data suggest that lyophilized FMT product given orally to patients with recurrent CDI could reduce subsequent infections (11, 12). However, there is an unmet need for lyophilized FMT product with fresh and frozen fecal FMT products, with follow-up of recipients for five months for clinical response and for one month after FMT for microbiome composition in a subset of subjects.

### SUMMARY OF THE INVENTION

Embodiments of the present disclosure provide compositions comprising human fecal microbiota and methods of use thereof. In a first embodiment, there is provided a composition comprising an extract of human feces comprising fecal microbiota and at least one sugar. In some aspects, the extract of human feces comprises viable fecal microbiota. In particular aspects, the human feces is initially derived from an individual screened to have a normal, healthy or wild type population of fecal flora. In some aspects, the extract of human feces is from a donor who has not been exposed to antibiotics for at least three months prior to sample collection.

In some aspects, the sugar is mannitol or sucrose. In certain aspects, the at least one sugar is present at a concentration of 0.5% to 5% (vol/vol), such as 1% to 3% (vol/vol). In some aspects, the at least one sugar is present at a concentration of at least 1%, 2%, 3%, 4%, 5%, 6%, 7%, 8%, 9%, or 10% (vol/vol). In particular aspects, the at least one sugar, such as mannitol, is present at a concentration of about 2% (vol/vol).

In certain aspects, the composition comprises at least 4 different phyla of bacteria selected from the group consisting of Bacteroidetes, Firmicutes, Proteobacteria, Verrucomicrobiae, and Actinobacteria. In some aspects, the composition comprises at least 5, 6, 7, 8, 9, or 10 different classes of bacteria selected from the group consisting of Actinobacteria, Bacteroidia, Bacilli, Clostridia, Erysipelotrichi, Alphaproteobacteria, Betaproteobacteria, Gammaproteobacteria, Mollicutes, and Verrucomicrobiae. In certain aspects, the composition comprises at least 4, 5, 6, 7, 8, 9, or 10 different families of bacteria selected from the group consisting of Lachnospiraceae, Enterobacteriaceae, Bacteroidaceae, Ruminococcaceae, Verrucomicrobiaceae, Bifidobacteriaceae, and Veillonellaceae. In additional aspects, the composition is enhanced with one or more additional bacterial species, such as those selected from the group consisting of a Bacteroides species, a Firmicutes species, and Bacillus thuringiensis.

In some aspects, the fecal microbiota essentially consists of particles that will pass through a sieve having a sieve size of 2.0 mm, 1.0 mm, 0.5 mm, 0.25 mm, 0.212 mm, 0.180 mm, 0.150 mm, 0.125 mm, 0.106 mm, 0.090 mm, 0.075 mm, 0.063 mm, 0.053 mm, 0.045 mm, 0.038 mm, 0.032 mm, 0.025 mm, 0.020 mm, or 0.01 mm. In some aspects, the composition comprises at least 1×10¹⁰, 1×10¹², 1.5×10¹², 2×10¹², 2.5×10¹², or more bacteria.

In certain aspects the composition is a formulation for oral administration, administration by nasogastric tube, or administration by colonoscopy. In particular aspects, the composition is formulated for oral administration, such as in capsules. In some aspects, the composition is lyophilized. In other aspects, the composition is frozen. In some aspects, the composition is a solid. In some aspects, the composition has been frozen or lyophilized for at least 1 week, 2 weeks, 3 weeks, one month, 2 months, 3 months, 6 months, or longer. In specific aspects, the composition is in the form of a tablet, a troche, or a capsule, particularly a capsule with an acid-resistant enteric coating. In some aspects, the coating comprises hypromellose (HPMC) or hypromellose phthalate (HPMCP). In certain aspects, the coating comprises a mixture of HPMC and HPMCP. In some aspects, the capsule is formulated to release its contents in the small intestines. In some aspects, the capsule has a volume of between 0.2 and 2 mls, between 0.2 and 1.5 mls or between 0.5 and 1.5 mls. In certain aspects, the composition is capable of being re-formulated for final delivery as comprising a liquid, a suspension, a gel, a geltab, a semisolid, a tablet, a sachet, a lozenge, a capsule, or as an enteral formulation. In some aspects, the composition is formulated for multiple administrations.

In some aspects, the composition has a water content of less than 5%. In certain aspects, the composition has a water content of less than 4%, 3%, 2%, 1%, or 0.5%. In some aspects, the composition is frozen. In some aspects, the composition is solid.

In additional aspects, the composition further comprises a saline, a defoaming agent, a surfactant agent, a lubricant, an acid neutralizer, a marker, a cell marker, a drug, an antibiotic, a contrast agent, a dispersal agent, a buffer or a buffering agent, a sweetening agent, a debittering agent, a flavoring agent, a pH stabilizer, an acidifying agent, a preservative, a desweetening agent, coloring agent, at least one vitamin, mineral supplement, a dietary supplement, a prebiotic nutrient or any combination thereof.

In some embodiments, the present disclosure provides an enteric-coated capsule comprising a lyophilized extract of human feces comprising at least 1×10¹⁰ viable fecal microorganisms, a sugar, and a salt, wherein the enteric-coated capsule is formulated to release its contents in the small intestines upon oral administration to a human subject. In some aspects, the sugar comprises mannitol. In further aspects, the sugar (e.g., mannitol) is present at about 0.5% to 5%, 1% to 5%, or 1% to 3%. In some aspects, the salt comprises sodium chloride. In certain aspects, the salt (e.g., sodium chloride) is present at about 0.1% to 2%, 0.25% to 1%, or 0.5% to 1%. In specific aspects, prior to lyophilization human feces are blended with a salt solution and a sugar, filtered to remove all solids larger than 0.5 mm, or frozen for at least one hour. In other aspects, prior to lyophilization human feces are blended with a salt solution and a sugar, filtered to remove all solids larger than 0.5 mm, and frozen for at least one hour. In some aspects, prior to lyophilization the human feces are blended with a saline solution at a volume ratio of from 1:2 to 1:10 (feces to saline) and mannitol to a final concentration of 0.5 to 5%. In specific aspects, prior to lyophilization human feces are blended with a saline solution at a volume ratio of 1:5 (feces to saline) and mannitol to a final concentration of 2%. In some aspects, the enteric coating comprises a mixture of HPMC and HPMCP.

In another embodiment, the composition is for therapeutic use to treat a patient having a disease characterized by dysfunctional microbiota. In some aspects, the disease is selected from the group consisting of a Clostridium difficile colitis, a metabolic syndrome, obesity, asthma, eczema, an eosinophilic disorder of the gastrointestinal tract, inflammatory bowel disease, irritable bowel syndrome, ulcerative colitis, Crohn's disease, enterohemorrhagic colitis, chronic diarrhea, chronic constipation, an eating disorder, malnutrition, rheumatoid arthritis, systemic lupus erythematosus, multiple sclerosis, fibromyalgia, chronic fatigue syndrome, nonalcoholic fatty liver disease, and a neurodegenerative disorder. In one particular aspect, the neurodegenerative disorder is Parkinson's disease. In specific aspects, the disease is a Clostridium difficile infection, such as acute Clostridium difficile colitis, relapsing Clostridium difficile colitis, or severe Clostridium difficile colitis.

In addition to the exemplified indication of recurrent CD, the disclosed compositions and methods are being used to treat or alleviate symptoms associated with Irritable Bowel Syndrome (IBS); Fatty Liver disease; and Parkinson's Disease. Additional indications for which these compositions and methods can be used include, but are not limited to, metabolic syndrome, diabetes, obesity, neurodegenerative disorders, neurodevelopmental disorders, autism, minimal hepatic encephalopathy, atherosclerosis, pancreatitis, fibromyalgia, autoimmune diseases or disorders, allergic diseases or disorders, asthma, eczema, an eosinophilic disorder of the gastrointestinal tract, rheumatoid arthritis, systemic lupus erythematosus, multiple sclerosis, fibromyalgia, chronic fatigue syndrome, nonalcoholic fatty liver disease.

A further embodiment provides a composition of the embodiments (e.g., an extract of human feces comprising fecal microbiota and at least one sugar) for therapeutic use to replace a patient's microbiota. In some aspects, some, most, or substantially all of the patient's colon, gut or intestinal microbiota are removed prior to the administering of the composition. In certain aspects, a single dosage of the composition comprises between 1×10¹⁰ and 5×10¹⁰ cells.

In another embodiment, there is provided a method of preparing a composition comprising an extract of human feces comprising fecal microbiota, the method comprising: blending a fecal sample from a fecal donor with a diluent, filtering the blended fecal sample, and adding at least one sugar to the blended fecal sample. In another embodiment, there is provided a method of preparing a composition comprising an extract of human feces comprising fecal microbiota, the method comprising: blending a fecal sample from a fecal donor with a diluent and at least one sugar and filtering the blended fecal sample. In particular aspects, the diluent is NaCl, such as 0.8% to 0.9% NaCl, particularly 0.85% NaCl. In some aspects, the blending is performed at a 1:2 to 1:10 dilution (fecal sample to diluent). In certain aspects, the blending is performed at a 1:4 to 1:6 dilution (fecal sample to diluent), such as a 1:5 dilution (fecal sample to diluent). In particular aspects, the diluent does not comprise an antibacterial preservative. In some aspects, the blending is performed using a paddle blender, e.g., Stomacher® 80 Master.

In certain aspects, filtering comprises passing the fecal sample through a sieve, such as sterile gauze. The sterile gauze may be composed of multiple layers, such as 1, 2, 3, 4, 5, 6, or more layers, particularly 5 layers. The sterile gauze may be moistened, such as with saline. In some aspects, the sieve comprises a sieve size of no greater than 2.0 mm, 1.0 mm, 0.5 mm, 0.25 mm, 0.212 mm, 0.180 mm, 0.150 mm, 0.125 mm, 0.106 mm, 0.090 mm, 0.075 mm, 0.063 mm, 0.053 mm, 0.045 mm, 0.038 mm, 0.032 mm, 0.025 mm, 0.020 mm, or 0.01 mm. In particular aspects, filtering is performed more than once, such as 2, 3, 4, or more times.

In further aspects, solids can be removed from the sample by sedimenting solids via centrifugation (e.g., at 500-3000×g). In some aspects, solids are removed from a sample by both filtering and centrifugation of the sample.

In some aspects, the at least one sugar is mannitol. In certain aspects, the at least one sugar is present at a concentration of 0.5% to 5% (vol/vol), such as 1% to 3% (vol/vol), particularly at a concentration of at least 1%, 2%, 3%, 4%, 5%, 6%, 7%, 8%, 9%, or 10% (vol/vol).

In additional aspects, the method further comprises centrifuging a filtrate from the filtering step. In some aspects, the method further comprises freezing or lyophilizing the composition. In certain aspects, the method further comprises reconstituting the composition with an aqueous solution.

In some aspects, the method further comprises freezing the composition for at least 30 minutes, 1 hour, 2 hours, 3 hours, or 4 hours. In some aspects, the sample is frozen for no more than 1 month, 1 week, 3 days, or 1 day. In some aspects, the method further comprises lyophilizing the frozen composition. In particular aspects, the method further comprises formulating the lyophilized composition in enteric capsules. In some aspects, the enteric capsules are acid resistant. In some aspects, the capsules comprise HPMC and HPMCP. In particular aspects, the capsules are Acid Resistant AR Caps® (see the world wide web at farmacapsulas.com/en/productos/ar-caps-2/). In specific aspects, the method further comprises storing the enteric capsules at between 10° C. and 1° C., such as at about 4° C.

A further embodiment provides a method for replacing, supplementing, or modifying a subject's colon microbiota, the method comprising administering to the subject a composition of the embodiments (e.g., an extract of human feces comprising fecal microbiota and at least one sugar). In some aspects, the method further comprises reconstituting the composition with an aqueous solution. In certain aspects, the method further comprises removal of some, most, or substantially all of the subject's colon, gut or intestinal microbiota prior to administering the composition. In some aspects, the composition is administered more than once. In certain aspects, the composition is administered daily, weekly, or monthly. In some aspects, the composition is administered for two, three, or four months. In certain aspects, the composition is administered orally or by colonoscopy.

In yet another embodiment, there is provided a method for treating a subject having a disease characterized by dysfunctional microbiota, the method comprising administering to the subject in need thereof an effective amount of the composition of the embodiments (e.g., an extract of human feces comprising fecal microbiota and at least one sugar). In some aspects, the method further comprises removal of some, most, or substantially all of the subject's colon, gut or intestinal microbiota prior to administering the composition. In certain aspects, the disease is selected from the group consisting of a Clostridium difficile colitis, a metabolic syndrome, obesity, asthma, eczema, an eosinophilic disorder of the gastrointestinal tract, inflammatory bowel disease, irritable bowel syndrome, ulcerative colitis, Crohn's disease, enterohemorrhagic colitis, chronic diarrhea, chronic constipation, an eating disorder, malnutrition, rheumatoid arthritis, systemic lupus erythematosus, multiple sclerosis, fibromyalgia, chronic fatigue syndrome, nonalcoholic fatty liver disease, and a neurodegenerative disorder. In some aspects, the neurodegenerative disorder is Parkinson's disease. In particular aspects, the Clostridium difficile colitis is recurrent. In some aspects, the composition has been frozen or lyophilized for at least 1 week, 2 weeks, 3 weeks, one month, 2 months, 3 months or 6 months prior to the administration.

In certain aspects, the composition is administered more than once. In some aspects, the composition is administered daily, weekly, or monthly. In certain aspects, the composition is administered for two, three, or four months. In specific aspects, the composition is administered orally or by colonoscopy.

In additional aspects, the further comprises evaluating the subject by microbiome sequencing. In some aspects, administering the composition results in increased microbial diversity in the subject. In certain aspects, administering the composition results in a decrease in the proportion of Proteobacteria and/or in increase in the proportion of Firmicutes, Actinobacteria, Bacteroidetes and/or Verrucomicrobia in the subject's microbiota.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%. Most preferred is a composition in which no amount of the specified component can be detected with standard analytical methods.

As used herein in the specification and claims, “a” or “an” may mean one or more. As used herein in the specification and claims, when used in conjunction with the word “comprising”, the words “a” or “an” may mean one or more than one. As used herein, in the specification and claim, “another” or “a further” may mean at least a second or more.

As used herein in the specification and claims, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating certain embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1B—Microbiome diversity (1A) and richness (1B) in fecal samples obtained from subjects with recurrent C. difficile infection who received fecal Microbiota Transplantation (FMT) before and after the procedure, as compared with the donors, expressed by Shannon Diversity Index (1A) and number of taxonomic units (1B). P values indicate three-group Kruskal Wallis rank test between FMT methods. Colors indicate three types of FMT products. Box plots shown with medians in the middle, with hinges at the first and the third quartile. Vertical whiskers are shown for 1.5 times inter-quartile range, and outliers are shown in dots outside the whisker ranges.

FIG. 2—Microbiota relative abundance at the phylum level for donors and FMT recipients grouped by administration route and treatment days before and after FMT.

FIG. 3—Microbiota relative abundance at the family level showing the most abundant taxonomic families for donors and FMT recipients grouped by administration route and treatment days before and after FMT.

FIG. 4—Principal coordinate analysis (PCoA) of fecal microbiome composition at each time point from 52 subjects with recurrent CDI treated with fresh, frozen or lyophilized fecal microbiota transplantation (FMT) and the 8 donors providing product that was given via colonoscopy, September 2013 to April 2016. 07d_FMT=stool obtained 7 days post FMT; 14d_pFMT=stool obtained 14 days post FMT; 30d_pFMT=stool obtained 30 days after FMT; b_FMT=baseline stool pre-FMT.

FIG. 5—Schematic of the Manufacturing and Packaging Procedure.

### DESCRIPTION OF ILLUSTRATIVE EMBODIMENTS

### I. The Present Embodiments

The present studies confirmed the activity of both frozen and lyophilized fecal product for fecal matter transplantation (FMT) in patients with recurrent Clostridium difficile infection (CDI) without obvious adverse experiences. Thus, the frozen and lyophilized product with a sugar, such as 2% mannitol, as a cryoprotectant is provided in the present disclosure. In particular, the fecal composition can be encapsulated in enteric-coated capsules for oral use. In addition to recurrent CDI infections, the fecal composition provided herein may be used for other disorders in which the subject has a dysfunctional microbiota.

### II. Summary of Pilot Studies

PRIM-DJ2727 (microbiota suspension) is an intestinal microbial suspension prepared from stool obtained from carefully and thoroughly screened healthy human donors. PRIM-DJ2727 capsules are administered orally. PRIM-DJ2727 is prepared from a standard amount of healthy human stool mixed with 0.85% NaCl (normal saline) and 2% mannitol. Product in capsule formulation is stored at the University of Texas School of Public Health at 4° C. The product is given to the subjects orally at the clinic with drinking water. PRIM-DJ2727 can be delivered, inter alia, by colonoscopy, retention enema, or oral administration of lyophilized capsules, comprising for example lyophilized product with 2% mannitol.

In a preferred embodiment, subjects/patients with multiple (more than 3) recurrent C. difficile infections (RCDI) can be treated with multiple or weekly oral administration of lyophilized and encapsulated PRIM-DJ2727 (5-6 capsules of microbiota suspension). RCDI patients treated with PRIM-DJ2727 are expected to demonstrate improved flora diversity and a reduction in the symptoms or RCDI, such as diarrhea, nausea, vomiting, abdomen pain, fever or other potential flora mediated disorders, as well as reductions in the presence of C. difficile toxins.

In another preferred embodiment, subjects/patients with Nonalcoholic fatty liver disease (NAFLD) can be treated with weekly oral administration of lyophilized and encapsulated PRIM-DJ2727 (5-6 capsules of microbiota suspension). It is believed that intestinal flora may play an active role in the development of insulin resistance and probiotics can have a favorable effect in the management of NAFLD in animals and humans, in some cases reducing proinflammatory cytokines. NAFLD patients receiving therapy are expected to improve with regards to metabolic function, as determined by blood studies; increasing BMI; changes in diabetic control; and liver function studies In addition, NAFLD patients receiving therapy are expected to experience decreased symptoms of fatty liver disease: feeling tired, loss of weight or appetite, weakness, nausea, confusion, poor judgement or trouble concentrating.

In yet another preferred embodiment, subjects/patients with Parkinson's Disease can be treated with weekly oral administration of lyophilized and encapsulated PRIM-DJ2727 (5-6 capsules of microbiota suspension). Abnormal microbiota, dysbiosis and small bowel bacterial overgrowth may contribute to the pathogenesis of Parkinson's disease through effects on cytokine levels of the gut, local mucosal inflammation, motility regulation and alteration of mucosal permeability and constipation. Parkinson's Disease patients receiving therapy are expected to improve with regards to bowel habits, as well as neurologic symptoms as measured on the Unified Parkinson's Disease Rating Scale which measures mentation, behavior, mood, activities of daily living and motor manifestations as well as changes to anti-PD medications and patient assessments of global improvement in PD and quality of life.

In yet another preferred embodiment, subjects/patients with Irritable Bowel Syndrome (IBS) can be treated with weekly oral administration of lyophilized and encapsulated PRIM-DJ2727 (5-6 capsules of microbiota suspension) for 8 weeks. Irritable Bowel Syndrome (IBS) patients receiving therapy are expected to improve with regards to bowel habits, symptoms such as, but not limited to, diarrhea, constipation, abdomen pain as well a reduction in overall IBS symptom severity scale (IBS-SSS questionnaire),

### III. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Randomized Clinical Trial: Fecal Microbiota Transplantation (FMT) for Recurrent Clostridium difficile Infection

72 subjects with ≥3 bouts of CDI were randomized in a double-blind study to receive fresh, frozen or lyophilized FMT product via colonoscopy from 50 g of stool per treatment from eight healthy donors. Recipients provided stools pre- and 7, 14 and 30 days post-FMT for C. difficile toxin and, in a subset, microbiome composition by 16S rRNA gene profiling.

Enrollment criteria included a history of ≥3 separate bouts of CDI in the past 12 months in non-pregnant adults ≥18 years of age. A study exclusion was previous receipt of an FMT. Subjects were instructed to stop all anti-CDI medications 48 hours before the FMT procedure. Subjects were enrolled and randomized by computer table to receive fresh, frozen or lyophilized donor fecal microbiota on a 1:1:1 allocation. The subjects were contacted daily by telephone after FMT for safety and clinical features and after one week contacted by telephone weekly for 5 months and instructed to report health issues. Subjects provided written subject diary of symptoms at regular intervals for five months after FMT for adverse events, stool pattern and CDI episodes. Recipients provided four stools each: 1-2 days before and 7, 14 and 30 days after FMT. When diarrhea occurred, the subjects underwent C. difficile toxin testing and if positive, antimicrobial treatment was given at the discretion of their physicians with the recommendation that they take two weeks of vancomycin followed by tapering doses of the drug.

Donors were thoroughly evaluated by history, serum and stool studies using previously published methods (13) and were required to have all negative results and to have normal body BMI.

Preparation of FMT Product—Individual stool samples from donors of ≥50 g were processed within 4 hours of passage, diluted in 0.85% NaCl (1:10) with a total volume of 1,500 mL and mixed in a Stomacher® 80 Master (Seward Laboratory System Inc., Davie, Fla.) in a sterilized bag, then filtered through moistened 5-layer sterile gauze in a funnel (both sterilized) under biological safety cabinet. Stools remained at room temperature less than 8 hours after which bacterial content would be expected to be reduced (14).

Fresh aliquots were used within 2 hours of preparation. Frozen aliquots were stored at −80° C. and used within 6 months after preparation. For the product to be lyophilized, the 50 g filtered solution was frozen at −80° C. for at least 6 hours, then placed under a Freeze Dry System (Labconco, Kansas City, Mo.) enabling the water in the product to vaporize without passing through the liquid phase (sublimation). After lyophilization, the product was maintained as powder at 4° C. in sealed 50 mL conical tubes and used within 6 months after preparation. Prior to the study the inclusion of the 10% glycerol with the fecal extract was evaluated but a dry product by lyophilization could not be obtained so no cryoprotectant was used in the study.

On the day of FMT, 250 mL of sterilized 0.85% NaCl (Thermo Fisher Scientific, Waltham, Mass.) was added to the vials of lyophilized donation products. After reconstitution, the product was kept at 4° C. and used within 4 hours.

Transplantation was performed via colonoscopy after polyethylene glycol colonoscopy bowel preparation the night before. Subjects took a single dose of 4 mg loperamide 3 hours before the procedure. Two gastroenterologists performed all FMT administrations infusing 50% of the product in the proximal colon with the remaining 50% infused in progressively decreasing amounts until reaching the rectum where the last portion of product was delivered.

Study Blinding—Only the laboratory director, was aware of the type of product assigned to subjects. Syringes with product prepared in the laboratory and identified by number were taken to the endoscopy suite at a university hospital in Houston for blind product administration. The treating gastroenterologist, the nurses following up the subjects and the recipients were unaware of the product assignment.

Laboratory Studies—All stools (donor and recipient) were examined for form and tested for presence of C. difficile toxin by commercial enzyme immunoassay (Remel, Lenexa, Kans.). Microbiota composition and relative abundance were evaluated by 16S rRNA gene compositional analysis in a subset of subjects. Using methods developed for the Human Microbiome Project, DNA isolation and microbiome sequencing were conducted at the Baylor College of Medicine's Alkek Center for Metagenomics and Microbiome Research (15, 16). Briefly, genomic bacterial DNA was extracted from fecal samples using the MO BIO PowerSoil DNA Isolation Kit (MO BIO Laboratories, Inc., Carlsbad, Calif.). One aliquot of each frozen stool sample was thawed, and 500 μL of stool was transferred to a MO BIO PowerSoil DNA Extraction PowerBead Tube. Samples were incubated at 95° C. for 10 minutes, then at 65° C. for 10 minutes, followed by genomic DNA extraction using the MO BIO PowerSoil DNA Extraction Kit protocol, and DNA samples were stored at −20° C.

Microbiome sequence analysis was performed on the first 52 subjects providing all four stool samples. DNA extraction, polymerase chain reaction and sequencing were performed using a published protocol (17). Briefly, the 16S rRNA gene V4 region was amplified by PCR and sequenced in the MiSeq platform (Illumina, San Diego, Calif.) using the 2×250 bp paired-end protocol yielding pair-end reads. The primers used for amplification contain adapters for MiSeq sequencing and single-end barcodes to permit pooling and direct sequencing of polymerase chain reaction products (17). Read pairs were demultiplexed based on the unique molecular barcodes, and reads were merged using USEARCH v7.0.1090 (18) allowing zero mismatches and a minimum overlap of 50 bases. Merged reads were trimmed at first base with Q5. In addition, a quality filter was applied to the resulting merged reads and reads containing more than 0.05 expected errors were discarded. 16S rRNA gene sequences were clustered into Operational Taxonomic Units at a similarity cutoff value of 97% using the UPARSE algorithm (19, 20). Operational Taxonomic Units were mapped to an optimized version of the SILVA Database (21) containing only the 16S v4 region to assign taxonomies. Demultiplexed reads were mapped to UPARSE Operational Taxonomic Units to recover abundances.

Statistical Methods—For quantitative description of changes in microbiota, diversity and richness of microbiota were first measured in each sample. Shannon diversity (Shannon entropy) (22) was used to measure diversity and total number of Operational Taxonomic Units with mapped reads to measure richness. Box plots were generated by grouping samples into five groups: donors, recipients before FMT, and recipients 7, 14, and 30 days post-FMT. Each sample group was then divided into the sub-groups of fresh, frozen, and lyophilized depending on the FMT delivery method. To determine whether significant differences in diversity or richness measures between different groups of samples existed, the Kruskal-Wallis test was used. The Kruskal-Wallis test is a non-parametric rank-based method to test whether samples in two or multiple groups are drawn from the same distribution without assuming a specific underlying distribution (23).

Relative abundances of each phylum and family were then aggregated relative to the donor product using stacked bar plots. In addition to diversity and richness measures, 2-dimensional plots of individual samples were generated to visualize relative similarity and discrepancy between individual samples. Beta microbiota diversity was used to compare the microbial composition between different samples by using the Bray-Curtis (BC) index in the Phyloseq (24) package with R version 3.3.1 for the PCoA analysis. All other analyses were performed in Stata version 14.

Sample Size Calculation—In developing a sample size, it was assumed each FMT treatment would be of comparable efficacy in preventing post-treatment recurrences. Because of ethical considerations wanting subjects to receive effective treatment, the Institutional Review Board approved a 100 subject study and in view of the large cost of the studies it was decided to perform an interim analysis after enrolling at least 20 subjects/group. When a statistical difference was found between two of the treatment groups enrollment was stopped.

Study Approval and Registry—The study used an approved Institutional Review Board protocol from the University of Texas Health Science Center and Baylor St. Luke's Medical Center (IRB # HSC-SPH-13-0119) and was registered in ClinicalTrials.gov (Number: NCT02318992).

Results

Of 11 potential donors screened, 8 (5 males and 3 females) were included in the study. Their median age was 58 years (range 36-77). For the 3 excluded donors, 2 were positive for fecal C. difficile toxin while for the third donor serum tested positive for serum hepatitis B surface antibody. Eight of the donors provided all stools needed for FMT products.

Seventy-three subjects were enrolled with one being lost to follow-up after FMT. Demographics and clinical features were similar in the 3 treatment groups (Table 1 below). Overall 63 of 72 (87%) subjects undergoing FMT achieved clinical wellness with no subsequent bouts of CDI during the two months following FMT. The cure rate was highest for the group randomized to receive fresh product, 25/25 (100%) and lowest for the group of subjects randomized to receive lyophilized product, 18/23 (78%), with intermediate response seen in the group receiving frozen product, 20/24 (83%) (p=0.041). The group receiving fresh product showed a statistically significant increase in efficacy compared with the group receiving lyophilized material (p=0.022); differences between groups receiving fresh vs. frozen did not reach significance (see Table 2 below). All failures of FMT occurred in the absence of receiving antibiotics during the observation period. One patient had recurrence of CDI at 150 days after FMT with lyophilized product and was considered cured in the analysis. The failures were all treated with a tapering dose of vancomycin and not followed further.

TABLE 1
Baseline characteristics of the recipients who
completed study with 5 months follow-up

| | | | | | Lyo- | | |
|---|---|---|---|---|---|---|---|
| | Fresh | | Frozen | | philized | | |
| | Microbiota | | Microbiota | | Microbiota | | P |
| Subject Variables | N = 25(%) | | N = 24 (%) | | N = 23 (%) | | value* |
| Female Gender | 21 | (84.0) | 18 | (75.0) | 13 | (56.5) | 0.10 |
| Median Age in | 75 | (19-97) | 62.5 | (33-88) | 63 | (20-87) | 0.26 |
| Years (Range) | | | | | | | |
| ≤3 CDI Episodes | 6 | (24.0) | 7 | (29.2) | 5 | (21.7) | 0.83 |
| before FMT | | | | | | | |
| >3 CDI Episodes | 19 | (76.0) | 17 | (70.8) | 18 | (78.3) | |
| before FMT | | | | | | | |
| Median Months | 2 | (0.5-10.5) | 1 | (0-9) | 1.5 | (0.5-6) | 0.17 |
| since last episode | | | | | | | |
| (Range) | | | | | | | |
| Pre-existent | 6 | (24.0) | 3 | (12.5) | 5 | (21.7) | 0.56 |
| Inflammatory | | | | | | | |
| Bowel | | | | | | | |
| Disease | | | | | | | |
| Pre-existent | 23 | (92.0) | 21 | (87.5) | 20 | (86.9) | 0.83 |
| comorbidities | | | | | | | |
| of heart, lungs, | | | | | | | |
| kidneys, central | | | | | | | |
| nervous system | | | | | | | |
| or multi-organ | | | | | | | |
| disease | | | | | | | |

*by X² test

TABLE 2
Clinical resolution of CDI in 72 subjects with recurrent
infection for 2 months after fecal microbiota transplantation.

| | | | | Comparisons p-value* (95% Confidence Interval) | | | |
|---|---|---|---|---|---|---|---|
| | | Cured+ | | All | Fresh vs | Fresh vs | Frozen vs |
| Group | N | (%) | | groups | Frozen | Lyophilized | Lyophilized |
| Fresh | 25 | 25 | (100) | 0.041 | 0.233 | 0.022 | 0.255 |
| Frozen | 24 | 20 | (83) | (0.32-245.10) | (1.37-405.41) | (1.59-415.41) | (0.89-9.82) |
| Lyophilized | 23 | 18 | (78) | | | | |
| Total | 72 | 63 | (87) | | | | |

*by X² test
+Cure diagnosed as freedom from bouts of CDI during the five months after FM

Twelve of 14 (86%) subjects with pre-existent inflammatory bowel disease were cured from their CDI. Six of the 10 recurrences of CDI occurred 7 days after FMT; one subject each was diagnosed with post-FMT CDI at 14 days, 41 days, 41 days and 150 days.

Cure rates in recipients were not significantly associated with specific donors (p=0.633). Cure rates for each donor were: 3/3 (100%), 2/3 (67%), 19/21 (90%), 5/6 (83%), 17/18 (83%), 7/8 (88%), 6/8 (75%), and 3/5 (60%).

Laboratory Studies—There was no difference in frequency of 2 month post-FMT CDI development between subjects with fecal C. difficile toxin at enrollment 3/11 (15%) compared with the toxin-negative group 6/61 (10%; p=0.108).

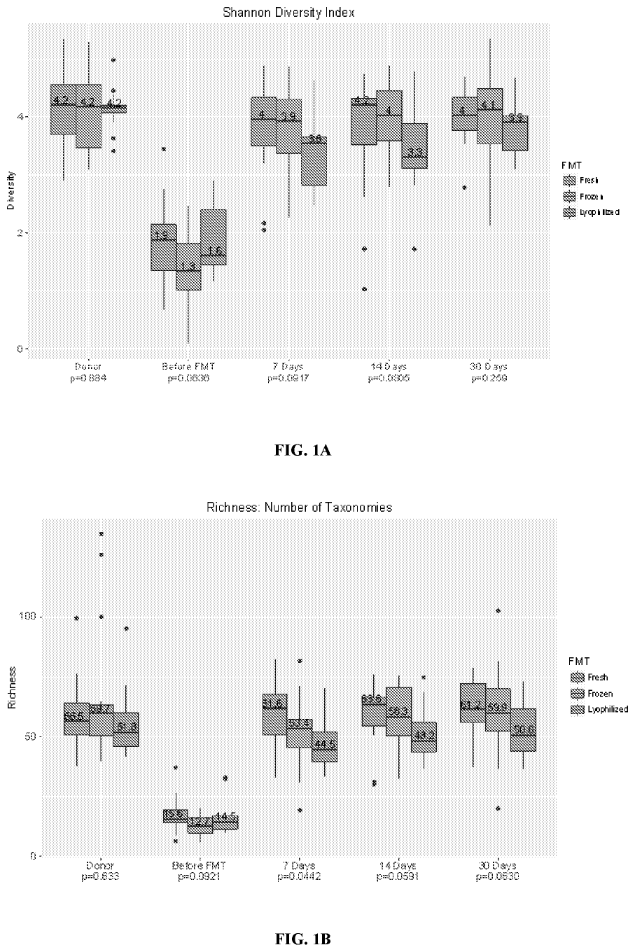

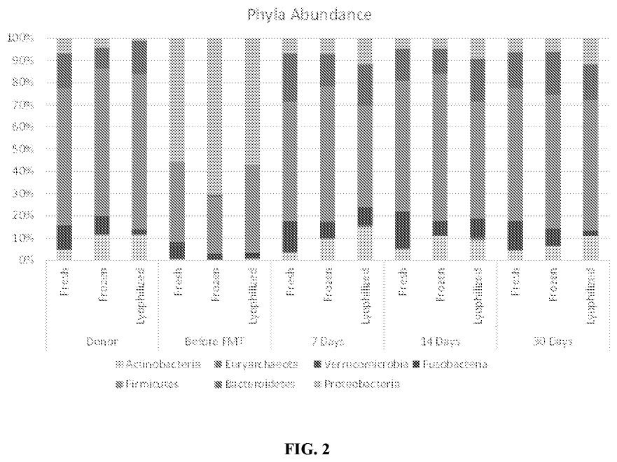

A subset of 52 subjects providing all four stool samples were included in this part of the study. These samples generated 1,558,302 high-quality, filtered, Illumina pair-end reads and were analyzed at a depth (rarefied) of 1,556 reads per sample. The mean/median diversity of fecal samples from recipients before FMT was notably lower than the average diversity of donors as measured by the Shannon diversity index (FIG. 1A). Recipient microbial diversity increased to essentially normal levels at 7 days after FMT and was maintained at 14 and 30 days after FMT. The differences between donors and recipients decreased with time. According to the Kruskal-Wallis analysis, diversity measures in all recipients tested were significantly lower than those of donors before FMT (p<2.2×10⁻¹⁶), after 7 days (p=0.0028), and after 14 days (p=0.0274), but they were not significantly lower after 30 days (p=0.0623). Recipients had significantly lower richness levels than donors before FMT (p<2.2×10⁻¹⁶) but not at 7, 14, and 30 days after FMT (p=0.225, 0.771, 0.310, respectively) (FIG. 1B).

Among the three FMT products, the Shannon diversity index was slightly lower for the group randomized to receive frozen product (P=0.0636). The median Shannon diversity indices 7 days post-FMT were lower in the lyophilized group compared with the fresh and frozen groups although three-way Kruskal-Wallis test statistic was not significant (p=0.0917). The median Shannon diversity index of the lyophilized group was 3.6, notably lower than that in the fresh and frozen groups (4.0 and 3.9, respectively). Diversity measures in the lyophilized group remained significantly lower at 14 days after FMT in a three-group test (p=0.0305). Thirty days after FMT, the median diversity in the lyophilized group increased to 3.9, similar to the other two groups (p=0.259). Total observed Operational Taxonomic Units in the lyophilized group remained significantly lower than other groups at 7, 14, and 30 days after FMT, but the median values gradually increased from 45 to 51 (FIG. 1B). Different time points within each treatment group were also statistically compared. All treatment groups show statistically significant improvements in both diversity and richness compared to the baseline of pre-FMT values (p<3×10⁻⁵). There was no statistically significant improvements in 14 days and 30 days measures compared to the 7 day data, except for the diversity measure of lyophilized group, where significant (p=0.027) improvement was observed in 30 days data compared to the 7 days (Supplementary Table S1).

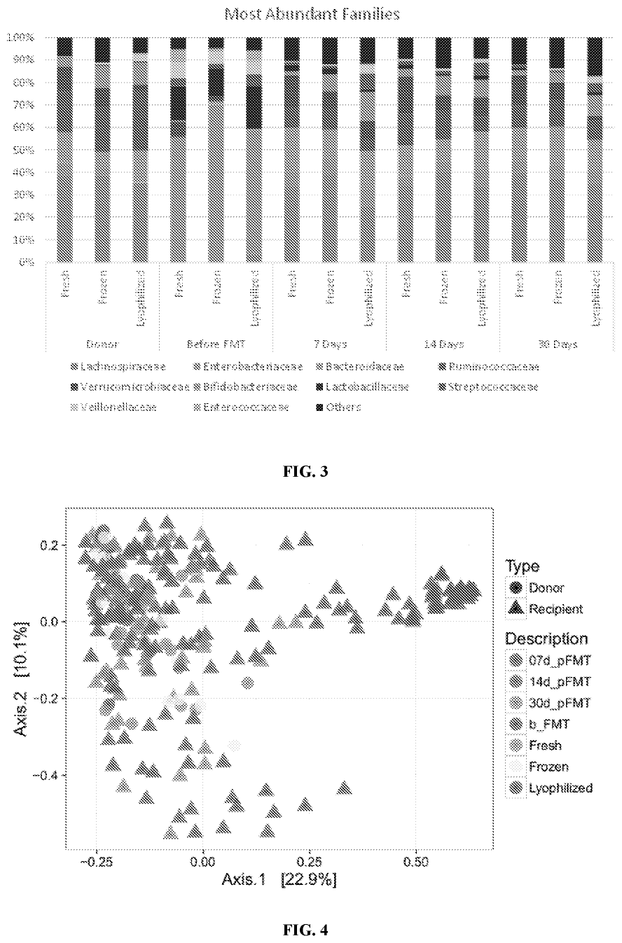

Taxonomic compositions of microbial population in subjects with recurrent CDI patients were also remarkably different from those of donors before FMT but resembled the composition of donors after FMT (FIGS. 2 and 3). Phylum-level relative abundances were first compared between subgroups (FIG. 2). In recipients before FMT, the most abundant phylum was Proteobacteria, accounting for more than 50% of the entire microbial population, while the same phylum represented only 10% of the relative abundances in donors. Recipients also had a higher composition of Fusobacteria than donors and relatively lower compositions of Actinobacteria and Firmicutes pre-FMT. After FMT, there were dramatic shifts in fecal microbiota with a decrease in Proteobacteria proportion and increases in Firmicutes, Actinobacteria, Bacteroidetes and Verrucomicrobia proportions. These changes were independent of which FMT product was used.

Post-FMT in the recipients, it was found that 10 microbial families represented more than 90% of the overall microbial population in all groups (FIG. 3) and significant redistributions of microbiota composition was also observed in subjects with recurrent CDI after FMT. At the family level, the increase of relative abundance in phylum Verrucomicrobia was mainly driven by Verrucomicrobiaceae, Bacteroidetes by Bacteroidaceae, and Actinobacteria by Bifidobacteriaceae, while the decrease in Proteobacteria was driven mostly by a decrease in Enterobacteriaceae. In phylum Firmicutes, decreased proportions of Enterococcaceae, Veillonellaceae, and Lactobacillaceae and increased proportions of Ruminococcaceae and Lachnospiraceae was observed. Increased abundance of these specific taxa in only certain number of recipients might have influenced these observations.

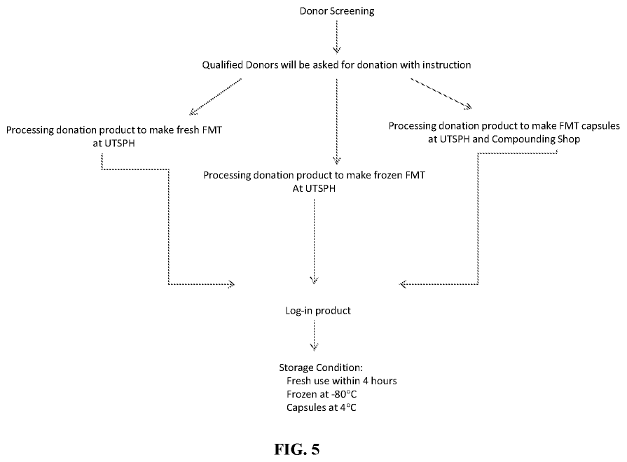

The microbiome composition of FMT recipients shifted over the study time points as demonstrated by PCoA of Bray-Curtis distances (FIG. 4). This analysis illustrates the important compositional changes in recipients following FMT. Fecal microbiota communities prior to FMT were highly distinct from fecal microbiota communities after FMT procedure, but the differences among the various FMT groups (fresh, frozen or lyophilized) at each time point did not show clear separation.

Adverse Events (AEs)—There were no serious adverse events. There were no observed differences in proportions of subject adverse events in the three groups. During the 48 hours after FMT a majority of subjects complained of nausea, mild diarrhea and transient abdominal discomfort (62/72, 86%). Two subjects experienced subjective fever, one at 2 days and the other 7 days after FMT; both of these failed FMT. Other complaints were fatigue (6/72, 8%), headache (4/72, 6%) and weight gain 2/72, 3%). One subject reported a gain of 7 pounds body weight 7 days after FMT. The other reported a gain of 8 pounds body weight 30 days after FMT, which brought their weight up to pre-CDI levels.

Overall resolution of CDI was 86% during 5 months of follow-up after FMT. Stool samples collected from subjects with CDI before FMT had significantly decreased bacterial diversity with a high proportion of Proteobacteria compared to donors. Cure rates were highest for the group receiving fresh product seen in 25/25 (100%), lowest for the lyophilized product 16/23 (78%; p=0.022 vs. fresh and 0.255 vs. frozen) and intermediate for frozen product 20/24 (p=0.233 vs fresh). Microbial diversity was reconstituted by day 7 in the subjects receiving fresh or frozen product. Improvement in diversity was seen by day 7 in those randomised to lyophilized material with reconstitution by 30 days.

Comparative efficacy in FMT was observed in subjects receiving fresh or frozen fecal product from the same donors. Lyophilized product with slightly lowered efficacy compared with fresh product resembled other treatments in microbial restoration one month after FMT.

### Example 2—Chemistry, Manufacturing, and Control

PRIM-DJ2727 is prepared from 150 g of human stool from a healthy, screened donor mixed with 750 mL 0.85% NaCl (normal saline) with 2% mannitol, which is added to reduce fluffiness of the product. It will contain normal microbiota. The product was designed and developed by the principal investigators from the University of Texas Health Science Center in Houston School of Public Health, University of Houston School of Pharmacy and Compounding Shop (License No. 29601) in Houston, Tex.

Donor Screening and Testing—Each donor must be disease- and enteric pathogen-free before donations begin. Donors are stable employees of the Texas Medical Center.

Since the product being used in the studies is derived from fecal matter obtained from a small number of volunteers, donor screening is critical to assure subject health to minimize safety concerns. A standard donor qualification process has been implemented. Donors sign an informed consent form and are screened at the Center for Infectious Disease, the University of Texas School of Public Health. Each donor must meet inclusion/exclusion criteria (see Table 3 below.

TABLE 3
Donor inclusion and exclusion criteria

| Inclusion | Exclusion |
|---|---|
| 1. Must be >18 years of age | 1. Tested positive for any of variables |
| 2. Able to provide and sign | mentioned below |
| informed consent | 2. History of autoimmune or atopic illness |
| 3. Able to complete and sign | or active cancer or ongoing immune |
| the donor questionnaire | modulating therapy |
| 4. Able to adhere to fecal | 3. First degree relative with intestinal |
| transplantation stool | carcinoma |
| collection requirements | 4. History of risk factors for acquisition of |
| within 6 months | HIV, syphilis, Hepatitis B, Hepatitis C, |
| | prion or any neurological disease as |
| | determined by the donor questionnaire |
| | 5 History of gastrointestinal disorder, e.g., |
| | inflammatory bowel disease (IBD), |
| | irritable bowel syndrome (IBS), |
| | chronic constipation or diarrhea, |
| | gastrointestinal malignancies |
| | 6. New sexual contacts during past |
| | 6 months |
| | 7. Tattoos, body piercing or incarceration |
| | 8 Major gastrointestinal surgical |
| | procedures |
| | 9. Antibiotic use during the preceding 3 |
| | months of donation |
| | 10. Drug or alcohol abuse |
| | 11. Fever >100.4° F. (38° C.) for the past |
| | 3 months |
| | 12. Signs or any symptoms, including |
| | persistent symptoms of communicable |
| | infection, including cold |
| | 13. A history of chronic pain syndromes |
| | (fibromyalgia, chronic fatigue) or |
| | neurologic, neurodevelopmental disorders |
| | 14. Receipt of any type of live vaccine |
| | within 3 months prior to stool donation |
| | 15. Current or previous medical or |
| | psychosocial condition |
| | 16. Metabolic syndrome, body mass index |
| | over 30 or moderate-to-severe |
| | undernutrition (Malnutrition) |
| | 17. Hospitalization during the preceding 3 |
| | months of donation |
| | 18. Regular attendance at outpatient |
| | medical or surgical clinics |
| | 19. International travel or recent medical |
| | tourism within 3 months period |

Potential donors complete a comprehensive initial health, family history and lifestyle questionnaire and then provide blood and stool sample for a comprehensive analysis for potential pathogens, including HIV, Hepatitis B, C and syphilis in the blood and a wide variety of pathogens in the stool. (see, Table 4 below)

TABLE 4
Donor testing

| | | Acceptance |
|---|---|---|
| Agent | Material | Criteria |
| Hepatitis B Core Antibody | Blood | Negative |
| Hepatitis B Surface Antigen | Blood | Negative |
| Hepatitis C Virus Antibody | Blood | Negative |
| Hepatitis A Virus IgM | Blood | Negative |
| HIV-1 and HIV-2 Antibody | Blood | Negative |
| Anti-HTLV I/II | Blood | Negative |
| Serologic Test for Syphilis | Blood | Negative |
| Clostridium difficile toxin A/B | Stool | Negative |
| Shigella spp. | Stool | Negative |
| Salmonella spp. | Stool | Negative |
| Campylobacter spp. | Stool | Negative |
| Shiga-toxin producing | Stool | Negative |
| Escherichia coli | | |
| Methicillin Resistant | Stool | Negative |
| Staphylococcus aureus | | |
| Vancomycin Resistant | Stool | Negative |
| Enterococcus spp. | | |
| Carbapenem Resistant | Stool | Negative |
| Enterobacteriaceas | | |
| Extended Spectrum β-lactanase | Stool | Negative |
| Producing E. coli | | |
| Aeromonas spp | Stool | Negative |
| Plesiomonas spp | Stool | Negative |
| Yersinia spp. | Stool | Negative |
| Vibrio spp | Stool | Negative |
| Cryptosporidum | Stool | Negative |
| Entamoeba histolytica | Stool | Negative |
| Cyclospora | Stool | Negative |
| Isospora | Stool | Negative |
| Rotavirus | Stool | Negative |
| Adenovirus | Stool | Negative |
| Norovirus | Stool | Negative |

FIG. 5 illustrates the manufacturing and packaging procedure from after donor screening is complete. Each donor must be disease- and enteric pathogen-free before donations begin. One day before donation, investigator contacts qualified donors to provide ≥200 grams of stool on the donation day. The donor is encouraged taking Docusate sodium (Colace) 200 mg in the evening before the procedure (single dose) and drink of plenty water. Supplies and instructions for sample collection are provided.

On the day of donation, 200 g of stool per transplant are obtained (all stools greater than 200 grams will be accepted and used in totality) with stool passage not more than 2 hours with ice package before submitting for processing. Stool will be kept in an Igloo cooler with ice pack during transportation, see the protocol # UT-SPH-CID-04-040A. Stools less than 200 g will be discarded. No more than 10 donation products will be made within each lot number. An additional stool from the same donor will be collected within one month screened for the second form of FMT.

Processing of the sample will be as follows:
Dilute stool samples (1:5 dilution) with sterilized 0.85% NaCl without antibacterial preservative.
Mix the above solution in a Stomacher® 80 Master (Seward Laboratory System Inc., Davie, Fla.) with sterilized bag. Initially, use the low setting until the sample breaks up, and then advance the speed gradually to the highest setting. Continue for 5 minutes until sample is smooth and homogeneous.
The suspension will be filtered using sterile gauze (5 layers) that has been moistened with saline and funnel (both are sterilized) under biological safety cabinet. Allow adequate time for slow filtration to end. NOTE: To expedite process, multiple funnel/filters can be used to combine product.
The filtration product will be centrifuged at 1500×g for 10 minutes

### Fresh Products

Number the samples as FMT-D-XXX-FRESH Intestinal Bacteria (at least 500 mL, Form 129-12-05)
Label the tubes with lot number (mm-dd-yyyy) and date and time processed
Fresh FMT donation products will be used within 4 hours

### Frozen Products

Number the samples as FMT-D-XXX-FROZEN Intestinal Bacteria (at least 500 mL, Form 129-12-05), sealed with plastic tubes (polyethylene or similar film)
Label the tubes with lot number (mm-dd-yyyy) and date and time processed
Add 2% mannitol microbiota cryoprotectant and store the frozen FMT products at −80° C. in room 804 RAS building (CID-0066-CAP)
Frozen FMT donation products can be stored for up to 6 months in a test tube rack clearly labelled with lot # and expiration date (6 months after preparation date)

### Lyophilized Products

Number the samples as FMT-D-XXX-LYOPHILIZED Intestinal Bacteria (at least 500 mL, Form 129-12-05)
Label the tubes with lot number (mm-dd-yyyy) and date and time processed
Add mannitol to a total weight of 2%
Freezing at −80° C. for at least 6 hours: The product (filtered intestinal bacteria in 50 mL tube) is frozen
After freezing, the product is placed under Freeze Dry System (Labconco, Kansas City, Mo.). This enables the frozen solvent in the product to vaporize without passing through the liquid phase, a process known as sublimation.
A lyophilized product (10 of 50 mL sterilized conical vials contains 100 grams of stool from a donor) must be sealed with parafilm, then will be taken to Compounding shop, 11851-A Wilcrest, Houston, Tex. 77031 (License No. 29601), to produce enteric coated capsules in batches of lyophilized product.

Storage—UTSPH-FMT-D-XXX-FRESH Intestinal Bacteria will be kept in an igloo color with ice pack and delivered to the procedure room and used within 4 hours after completion the process. UTSPH-FMT-D-XXX-FROZEN Intestinal Bacteria will be kept at −80° C. for up to 6 months at the University of Texas Health Science Center at Houston School of Public Health room 804 RAS (CID-0066-CAP). UTSPH-FMT-D-XXX-Lyophilized products containing bacteria from ≥200 g stool will be kept in a sterilized vial (50 mL conical tube) at 4° C. at the University of Texas Health Science Center at Houston School of Public Health room 320 RAS (CID-0068-CAP) for up to 6 months in a test tube rack clearly labelled with the lot # and expiration date. This FMT product will be delivered via colonoscopy. On the day of FMT, 500 mL of sterilized 0.85% NaCl (Thermo Fisher Scientific, Waltham, Mass.) will be added to the sterilized vials with lyophilized donation products (concentrated from 100 g stool). After reconstitution, the product will be used with 4 hours and kept at 4° C. During the transportation (15 minutes driving distance), the product will be kept in an igloo color with ice pack. The process for reconstitution will be completed at the University of Texas Science Center at Houston School of Public Health in room 320 RAS. UTSPH-FMT-D-XXX-Lyophilized Capsules containing bacteria from 100 g stool will be kept in a sterilized vial at 4° C. at the University of Texas Health Science Center at Houston School of Public Health room 320 RAS (CID-0068-CAP) for up to 6 months in a test tube rack clearly labelled with the lot # and expiration date. Upon completion of the process, stool samples must be recorded into the Donor Specimens Log.

Aside from the addition of 0.85% NaCl and 2% mannitol (Fisher Scientific), which is added to increase the density of the sample during processing as described above, the sample is not manipulated (i.e., no strains are purified from the sample.)

PRIM-DJ2727 (intestinal flora suspension) is obtained from healthy, screened donors. It contains normal microbiota. PRIM-DJ2727 will be labeled with lot number and date and time processed in addition with D-001-LIB-R-001.

Placebo will be identical to the investigational product but will not contain intestinal bacteria. Placebo will consist of Lactose (spray-dried USP 64.385 gm), and food color: powdered Black (0.847 gm), Brown (3.384 gm), and Yellow (3.384 gm); in the enteric capsules (size 00). Placebo will be made at Compounding shop, 11851-A Wilcrest, Houston, Tex. 77031 (License No. 29601).

Placebo products will be tested for bioburden testing according FDA standards regarding the microbial examination of non-sterile products, see Protocol # UT-SPH-CID-04-039A.

Environmental Assessment—PRIM-DJ2727 (intestinal flora suspension) is obtained from healthy, screened donors. It will contain normal fecal microbiota which produces no harmful effects to the environment, including air, soil or water. Since donors will be carefully screened, there should be no known enteric pathogens in the product (PRIM-DJ2727) with current knowledge and laboratory technology.

### Example 3—Prospective Randomized Pilot Study: Fecal Microbiota Transplantation (FMT) for Recurrent Irritable Bowel Syndrome

A double-blind, placebo-controlled, randomized 2:1 pilot study evaluating the effects of lyophilized PRIM-DJ272 microbiota on bowel function will be performed with randomly selected individuals suffering from irritable bowel syndrome (IBS). IBS is defined as following Rome III criteria IBS, having abdominal pain intensity with a weekly average of worst daily (in past 24 hours) abdominal pain score of ≥3.0 on a 0 to 10 point pain scale. Moderate to severe IBS is defined by a score of ≥175 overall on the IBS symptom severity scale (IBS-SSS).

Studies have shown that fecal flora disturbances occur in IBS. This pilot study is intended to characterize the microbiome in this group of subjects, and evaluate microbiota replacement treatment as a means of flora restoration. This study will evaluate the effects of lyophilized PRIM-DJ2727 microbiota on bowel function, as well as on abdominal pain in IBS subjects. Further, it will characterize the intestinal flora in subjects with IBS and determine the safety and trend in individuals with 50% improvements in diversity of fecal microbiome following administration of lyophilized PRIM-DJ2727 microbiota.

This study will compare the effect of treatment with lyophilized PRIM-DJ2727 microbiota to placebo, and evaluate its effect in individuals on the overall IBS symptom severity scale (IBS-SSS questionnaire) and a health survey questionnaire (SF-36), as well as evaluate the product safety of PRIM-DJ2727 given orally each week for 8 weeks, including whether the patient suffers from diarrhea, constipation, or abdomen pain. 45 individuals will be included, 30 treated with FMT and 15 with placebo.

This study will also characterize the microbiome (diversity and genera) and microbiome stability in subjects with IBS treated with fecal microbiota transplantation (FMT) for 8 weeks during an 8 month study. This study will compare the proportions of subjects who have adequate relief of global IBS symptoms for at least 2 weeks during the 8 weeks of therapy in the lyophilized PRIM-DJ2727 microbiota group and the placebo group, and compare the effect of lyophilized PRIM-DJ2727 microbiota on the stool form (Bristol Stool Scale) and stool frequency. Additionally, the study will compare the proportions of subjects who experience a decrease in the weekly average of worst abdominal pain in the past 24 hours score of at least 30% compared with baseline in the lyophilized PRIM-DJ2727 microbiota group and the placebo group.

The study will consist of 3 parts: the baseline, treatment and safety assessment periods. Subjects will have a screening evaluation visit with a study physician to include history and physical exam, a review of medical records, and any necessary screening tests required for inclusion if ordered. If the subject qualifies, their physician will refer the subject to a research coordinator at Kelsey-Seybold Clinic who will consent the subject, draw blood serum, provide a stool sample collection kit for the subject, and, if applicable, a urine pregnancy test.

After consent, subjects begin the 2-week run-in period. During the screening period (Days 1-14), participants will complete daily diary questionnaires as well as SF36 and IBS-SSS at the end of each week. An aliquot (2 mL) of each stool sample collected will be stored at −80° C. for future analysis (e.g. microbiome studies). The serum sample will be stored for future study, possibly related to cytokine and chemokine levels.

After completion of the screening phase, the eligible participants will be randomized to 8 weekly treatments of either lyophilized PRIM-DJ2727 microbiota or placebo. Treatment will be administered as twice filtered fecal microbiota product diluted in saline to 250 mL from a screened healthy donor, lyophilized and contained within enteric-coated capsules (5-6 capsules) in a weekly dose for 8 consecutive weeks. In the treatment phase, participants will continue to keep daily diaries for all bowel movements. The IBS-SSS and SF-36 and Hospital Anxiety and Depression Scale (HADS) questionnaires will be completed on the day of enrollment and at all evaluation appointments, including the end of study evaluation. Compliance and adverse effects also will be monitored through the daily diaries that are collected weekly. Follow up for each subject will be 6 months.

Based on 90% clinical efficacy in curing recurrent Clostridium difficile infection (CDI) with the lyophilized product, it is hypothesized there will be a 50% improvement in subjects with IBS treated with weekly FMT, compared to the placebo group. The 50% improvement will be determined by analyzing patients' scores on the IBS Symptom Severity Scale (IBS-SSS), the Health Related Quality of Life SF36 Questionnaire (SF36), and the Hospital Anxiety and Depression Scale (HADS). Using the 2:1 allocation ratio, it is expected that there is more than 80% power to detect the statistical difference when recruiting a total of 45 subjects (30 with FMT and 15 with placebo) in this preliminary study. Failure of the treatment is defined as a subject that has less than 50% improvement at the end of study.

To ensure safe study monitoring during the study, an independent, non-blinded, external Data and Safety Monitoring Board (DSMB) will be established to perform safety evaluations on an ongoing basis. A DSMB charter will be developed and will detail the review of the safety data. The schedule of assessments and procedures is provided in Section 9.0. The study will be discontinued if the DSMB identifies a trend of more severe illness in the active study participants.

The inclusion and exclusion criteria for this study are presented below, in Table 5.

TABLE 5
inclusion and exclusion criteria for IBS study.

| | | |
|---|---|---|
| INCLUSION | 1. | Must be a U.S. resident between the ages |
| CRITERIA | | of 18 and 70 years of age |
| | 2. | Able to speak, read and write in English |
| | | (to provide consent and complete |
| | | questionnaires) |
| | 3. | Willing to sign an informed consent form |
| | 4. | Have stable gastrointestinal disease not |
| | | requiring frequent changes in treatment |
| | 5. | Be diagnosed with Rome III criteria IBS |
| | | plus abdominal pain intensity >3 and |
| | | IBS-SSS score >175 |
| | 6. | Other GI conditions must be ruled out |
| | | including: celiac disease; chronic parasitic |
| | | infection; inflammatory bowel disease; or |
| | | other anatomical and structural |
| | | pathologies of the intestine |
| | 7. | Sexually active male and female subjects |
| | | of child-bearing potential must agree to |
| | | use an effective method of birth control |
| | | during the treatment and follow-up period |
| | | 8. Females of child-bearing potential must |
| | | have a negative pregnancy test |
| | | in the 72 hours before the procedure |
| EXCLUSION | 1. | Females who are pregnant or breastfeeding, |
| CRITERIA | | or planning to become pregnant |
| | | in the next 3 months |
| | 2. | Chronic parasitic infection |
| | 3. | Unstable bowel disease requiring changing |
| | | treatment, including known |
| | | inflammatory bowel disease |
| | 4. | Structural or metabolic diseases/conditions |
| | | that affect the gastrointestinal |
| | | system-celiac disease |
| | 5. | Significant medical condition: cardiovascular, |
| | | respiratory, renal, hepatic, gastrointestinal, |
| | | hematological, neurological, psychiatric, |
| | | or other disease that the study physician |
| | | thinks will interfere with the objectives |
| | | of the study |
| | 6. | Current or planned use of antibiotics with |
| | | expected activity against enteric |
| | | bacteria or current or expected use or |
| | | treatment with probiotics |
| | 7. | Current symptoms of severe depression, |
| | | as measured by HAD S score 15 |
| | 8. | Active alcoholism or substance abuse |
| | 9. | Subjects with abnormal results for HIV |
| | 10. | Subjects with abnormal results for hepatitis |
| | | B or C |
| | 11. | Participation in another clinical trial |
| | | (within last 30 days) |
| | 12. | Subjects with constipation predominant IBS |

### Example 4—Prospective Randomized Pilot Study: Fecal Microbiota Transplantation (FMT) for Parkinson's Disease

A double-blind, placebo-controlled, randomized 2:1 pilot study evaluating the effects of lyophilized PRIM-DJ272 microbiota on bowel function will be performed with randomly selected individuals suffering from Parkinson's Disease (PD).

Limited studies have shown that fecal flora disturbances occur in PD. This pilot study will completely characterize the microbiome in this group of subjects, and evaluate microbiota replacement treatment as a means of flora restoration in PD subjects. This study will characterize the intestinal flora in subjects with PD and determine safety and trends in improvements in diversity of colonic microbiome following administration of PRIM-DJ2727.

This study will examine improvements in flora diversity by oral administration of a fecal suspension from healthy donors, determining said improvements by comparing data from treated individuals with untreated controls. The study will also seek to determine whether there is improvement in bowel patterns in PD subjects, focusing on bowel habits following treatment.

Additionally, the study seeks to understand the neurological state of PD subjects as a result of fecal microbiome changes. The study will look at improvement in neurologic picture using the Unified Parkinson's Disease Rating Scale measuring mentation, behavior, mood, activities of daily living and motor manifestations as a result of PRIM-DJ2727 treatment. The subjects will also be monitored for change in required anti-PD medications following PRIM-DJ2727 treatment. The study will also ask subjects for assessments of global improvement in PD, as well as quality of life.

Individuals in the study will be carefully monitored and assessed for complications of PD including worsening of symptoms or other potential flora-mediated disorders. These data will be collected through the 16 weeks of treatment and an additional 8 weeks after treatment via in-clinic assessment. The study would seek to provide data that will be used to determine appropriateness of designing a properly powered clinical trial of microbial restoration treatment in this population.

Forty-five eligible subjects with Parkinson's disorder will be randomly assigned to receive either PRIM-DJ2727 in orally administered enteric coated capsules (30 subjects) or placebo capsules (15 subjects). Subjects meeting inclusion criteria will be randomized in a 2:1 ratio and given either active or placebo fecal microbial transplant and treated weekly for 16 weeks. A physical examination will be performed at the time of enrollment and both stool and blood samples will be collected. Stool and serum will also be collected during the run-in period, as well as during weekly treatments. Subjects will not be required to follow a special diet during this study. 2 mL of each stool sample collected will be stored at −80° C. for future analysis such as microbiome studies. Inclusion and exclusion criteria are listed below, in Table 6.

TABLE 6
Inclusion and Exclusion criteria for PD study

| | | |
|---|---|---|
| INCLUSION | 1. | All subjects must be ≥18 years of age |
| CRITERIA | 2. | Sexually active male and female subjects |
| | | of child-bearing potential must agree to |
| | | use an effective method of birth control |
| | | during the treatment and follow-up period |
| | 3. | Female subjects of child-bearing potential |
| | | must have a negative pregnancy test |
| | | in the 72 hours before the procedure |
| | 4. | Subject willing to sign an informed |
| | | consent form |
| | 5. | Subject deemed likely to survive |
| | | for ≥1 year after enrollment |
| | 6. | Subject diagnosed with PD |
| | 7. | Subject's attending physician will refer |
| | | and provide non-transplant care |
| | | for the subject |
| | 8. | Subject agrees to continue other |
| | | Parkinson's medication and |
| | | treatment efforts |
| EXCLUSION | 1. | Greater than 20 grams of ethanol |
| CRITERIA | | intake daily |
| | 2. | HIV positive |
| | 3. | HCV, HBV positive |
| | 4. | Other immune disorder or clinical |
| | | immunosuppression |
| | 5. | Probiotic must not be used during |
| | | study period |
| | 6. | Severe underlying disease such that the |
| | | subject is not expected to survive |
| | | for one or more years or unstable medical |
| | | condition requiring daily change in |
| | | treatments |
| | 7. | Current receipt of an antibiotic with |
| | | expected activity against enteric |
| | | bacteria |

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Seekatz A M, Rao A M, Santhosh K, Young V B. Dynamics of the fecal microbiome in patients with recurrent and nonrecurrent Clostridium difficile infection. Genome medicine. 2016; 8(1):47. PubMed PMID: 27124953.
2. Milani C, Ticinesi A, Gerritsen J, Nouvenne A, Lugli G A, Mancabelli L, et al. Gut microbiota composition and Clostridium difficile infection in hospitalized elderly individuals: a metagenomic study. Scientific reports. 2016; 6:25945. PubMed PMID: 27166072. Pubmed Central PMCID: 4863157.
3. Bakken J S, Borody T, Brandt L J, Brill J V, Demarco D C, Franzos M A, et al. Treating Clostridium difficile infection with fecal microbiota transplantation. Clinical gastroenterology and hepatology: the official clinical practice journal of the American Gastroenterological Association. 2011 December; 9(12):1044-9. PubMed PMID: 21871249. Pubmed Central PMCID: 3223289.
4. Brandt L J. Fecal transplantation for the treatment of Clostridium difficile infection. Gastroenterology & hepatology. 2012 March; 8(3):191-4. PubMed PMID: 22675283. Pubmed Central PMCID: 3365524.
5. Hamilton M J, Weingarden A R, Sadowsky M J, Khoruts A. Standardized frozen preparation for transplantation of fecal microbiota for recurrent Clostridium difficile infection. Am J Gastroenterol. 2012 May; 107(5):761-7. PubMed PMID: 22290405.
6. Kelly C R, de Leon L, Jasutkar N. Fecal microbiota transplantation for relapsing Clostridium difficile infection in 26 patients: methodology and results. Journal of clinical gastroenterology. 2012 February; 46(2):145-9. PubMed PMID: 22157239.
7. Manila E, Uusitalo-Seppala R, Wuorela M, Lehtola L, Nurmi H, Ristikankare M, et al. Fecal transplantation, through colonoscopy, is effective therapy for recurrent Clostridium difficile infection. Gastroenterology. 2012 March; 142(3):490-6. PubMed PMID: 22155369.
8. Lee C H, Steiner T, Petrof E O, Smieja M, Roscoe D, Nematallah A, et al. Frozen vs Fresh Fecal Microbiota Transplantation and Clinical Resolution of Diarrhea in Patients With Recurrent Clostridium difficile Infection: A Randomized Clinical Trial. JAMA. 2016 Jan. 12; 315(2):142-9. PubMed PMID: 26757463.
9. Youngster I, Russell G H, Pindar C, Ziv-Baran T, Sauk J, Hohmann E L. Oral, capsulized, frozen fecal microbiota transplantation for relapsing Clostridium difficile infection. JAMA. 2014 Nov. 5; 312(17):1772-8. PubMed PMID: 25322359.
10. Satokari R, Manila E, Kainulainen V, Arkkila P E. Simple faecal preparation and efficacy of frozen inoculum in faecal microbiota transplantation for recurrent Clostridium difficile infection—an observational cohort study. Aliment Pharmacol Ther. 2015 January; 41(1):46-53. PubMed PMID: 25355279.
11. Hecker M T, Obrenovich M E, Cadnum J L, Jencson A L, Jain A K, Ho E, et al. Fecal microbiota transplantation by freeze-dried oral capsules of recurrent Clostridium difficile infection. Open Forum Infectious Diseases. 2016; 3:10.1093/ofid/ofw091. Epub May 5, 2016.
12. Tian H, Ding C, Gong J, Wei Y, McFarland L V, Li N. Freeze-dried, Capsulized Fecal Microbiota Transplantation for Relapsing Clostridium difficile Infection. J Clin Gastroenterol. 2015 July; 49(6):537-8. PubMed PMID: 25955501.
13. van Nood E, Vrieze A, Nieuwdorp M, Fuentes S, Zoetendal E G, de Vos W M, et al. Duodenal infusion of donor feces for recurrent Clostridium difficile. N Engl J Med. 2013 Jan. 31; 368(5):407-15. PubMed PMID: 23323867.
14. Ott S J, Musfeldt M, Timmis K N, Hampe J, Wenderoth D F, Schreiber S. In vitro alterations of intestinal bacterial microbiota in fecal samples during storage. Diagn Microbiol Infect Dis. 2004 December; 50(4):237-45. PubMed PMID: 15582296.
15. Human Microbiome Project C. Structure, function and diversity of the healthy human microbiome. Nature. 2012 Jun. 13; 486(7402):207-14. PubMed PMID: 22699609. Pubmed Central PMCID: 3564958.
16. Human Microbiome Project C. A framework for human microbiome research. Nature. 2012 Jun. 13; 486(7402):215-21. PubMed PMID: 22699610. Pubmed Central PMCID: 3377744.
17. Caporaso J G, Lauber C L, Walters W A, Berg-Lyons D, Huntley J, Fierer N, et al. Ultra-high-throughput microbial community analysis on the Illumina HiSeq and MiSeq platforms. The ISME journal. 2012 August; 6(8):1621-4. PubMed PMID: 22402401. Pubmed Central PMCID: 3400413.
18. Edgar R C. Search and clustering orders of magnitude faster than BLAST. Bioinformatics. 2010 Oct. 1; 26(19):2460-1. PubMed PMID: 20709691.
19. Edgar R C. UPARSE: highly accurate OTU sequences from microbial amplicon reads. Nat Meth. 2013 10//print; 10(10): 996-8.
20. Edgar R C. UPARSE: highly accurate OTU sequences from microbial amplicon reads. Nature methods. 2013 October; 10(10):996-8. PubMed PMID: 23955772.
21. Quast C, Pruesse E, Yilmaz P, Gerken J, Schweer T, Yarza P, et al. The SILVA ribosomal RNA gene database project: improved data processing and web-based tools. Nucleic acids research. 2013 January; 41 (Database issue):D590-6. PubMed PMID: 23193283. Pubmed Central PMCID: 3531112.
22. Li K, Bihan M, Yooseph S, Methe B A. Analyses of the microbial diversity across the human microbiome. PLoS One. 2012; 7(6):e32118. PubMed PMID: 22719823. Pubmed Central PMCID: 3374608.
23. Theodorsson-Norheim E. Kruskal-Wallis test: BASIC computer program to perform nonparametric one-way analysis of variance and multiple comparisons on ranks of several independent samples. Computer methods and programs in biomedicine. 1986 August; 23(1):57-62. PubMed PMID: 3638187.
24. McMurdie P J, Holmes S. phyloseq: an R package for reproducible interactive analysis and graphics of microbiome census data. PLoS One. 2013; 8(4):e61217. PubMed PMID: 23630581. Pubmed Central PMCID: 3632530.
25. Li Y T, Cai H F, Wang Z H, Xu J, Fang J Y. Systematic review with meta-analysis: long-term outcomes of faecal microbiota transplantation for Clostridium difficile infection. Aliment Pharmacol Ther. 2016 February; 43(4):445-57. PubMed PMID: 26662643.
26. Costello S P, Conlon M A, Vuaran M S, Roberts-Thomson I C, Andrews J M. Faecal microbiota transplant for recurrent Clostridium difficile infection using long-term frozen stool is effective: clinical efficacy and bacterial viability data. Aliment Pharmacol Ther. 2015 Aug. 11. PubMed PMID: 26264455.
27. Khanna S, Montassier E, Schmidt B, Patel R, Knights D, Pardi D S, et al. Gut microbiome predictors of treatment response and recurrence in primary Clostridium difficile infection. Aliment Pharmacol Ther. 2016 October; 44(7):715-27. PubMed PMID: 27481036. Pubmed Central PMCID: 5012905.
28. Seekatz A M, Aas J, Gessert C E, Rubin T A, Saman D M, Bakken J S, et al. Recovery of the gut microbiome following fecal microbiota transplantation. mBio. 2014; 5(3):e00893-14. PubMed PMID: 24939885. Pubmed Central PMCID: 4068257.
29. Barcenilla A, Pryde S E, Martin J C, Duncan S H, Stewart C S, Henderson C, et al. Phylogenetic relationships of butyrate-producing bacteria from the human gut. Appl Environ Microbiol. 2000 April; 66(4):1654-61. PubMed PMID: 10742256. Pubmed Central PMCID: 92037.
30. Duncan S H, Barcenilla A, Stewart C S, Pryde S E, Flint H J. Acetate utilization and butyryl coenzyme A (CoA):acetate-CoA transferase in butyrate-producing bacteria from the human large intestine. Appl Environ Microbiol. 2002 October; 68(10):5186-90. PubMed PMID: 12324374. Pubmed Central PMCID: 126392.
31. Reeves A E, Koenigsknecht M J, Bergin I L, Young V B. Suppression of Clostridium difficile in the gastrointestinal tracts of germfree mice inoculated with a murine isolate from the family Lachnospiraceae. Infect Immun. 2012 November; 80(11):3786-94. PubMed PMID: 22890996. Pubmed Central PMCID: 3486043.
32. Gough E, Shaikh H, Manges A R. Systematic review of intestinal microbiota transplantation (fecal bacteriotherapy) for recurrent Clostridium difficile infection. Clin Infect Dis. 2011 November; 53(10):994-1002. PubMed PMID: 22002980. Epub 2011/10/18. eng.
33. Louie T J, Cannon K, O'Grady H, Wu K, Ward L. Fecal microbiota transplantation (FMT) via oral fecal microbial capsules for recurrent Clostridium difficile infection (rCDI), abstract 89. 2013.
34. Strasser S, Neureiter M, Geppl M, Braun R, Danner H. Influence of lyophilization, fluidized bed drying, addition of protectants, and storage on the viability of lactic acid bacteria. J Appl Microbiol. 2009 July; 107(1):167-77. PubMed PMID: 19302330.

What is claimed is:

1. A composition comprising (a) an extract of human feces comprising viable fecal microbiota and (b) at least one sugar.

2. The composition of claim 1, wherein the sugar is mannitol or sucrose.

3. The composition of claim 1, wherein the at least one sugar is present at a concentration of 0.5% to 5% (vol/vol).

4. The composition of claim 1, wherein the at least one sugar is present at a concentration of 1% to 3% (vol/vol).

5. The composition of claim 1, wherein the at least one sugar is present at a concentration of at least 1%, 2%, 3%, 4%, 5%, 6%, 7%, 8%, 9%, or 10% (vol/vol).

6. The composition of claim 1, wherein the composition comprises at least 4 different phyla of bacteria selected from the group consisting of Bacteroidetes, Firmicutes, Proteobacteria, Verrucomicrobiae, and Actinobacteria.

7. The composition of claim 1, wherein the composition comprises at least 5, 6, 7, 8, 9, or 10 different classes of bacteria selected from the group consisting of Actinobacteria, Bacteroidia, Bacilli, Clostridia, Erysipelotrichi, Alphaproteobacteria, Betaproteobacteria, Gammaproteobacteria, Mollicutes, and Verrucomicrobiae.

8. The composition of claim 1, wherein the composition comprises at least 4, 5, 6, 7, 8, 9, or 10 different families of bacteria selected from the group consisting of Lachnospiraceae, Enterobacteriaceae, Bacteroidaceae, Ruminococcaceae, Verrucomicrobiaceae, Bifidobacteriaceae, and Veillonellaceae.

9. The composition of claim 1, wherein the composition is enhanced with one or more additional bacterial species.

10. The composition of claim 9, wherein the one or more additional bacterial species are selected from the group consisting of a Bacteroides species, a Firmicutes species, and Bacillus thuringiensis.

11. The composition of claim 1, wherein the human feces is initially derived from an individual screened to have a normal, healthy or wild type population of fecal flora.

12. The composition of claim 1, wherein the fecal microbiota essentially consists of particles that will pass through a sieve having a sieve size of 2.0 mm, 1.0 mm, 0.5 mm, 0.25 mm, 0.212 mm, 0.180 mm, 0.150 mm, 0.125 mm, 0.106 mm, 0.090 mm, 0.075 mm, 0.063 mm, 0.053 mm, 0.045 mm, 0.038 mm, 0.032 mm, 0.025 mm, 0.020 mm, or 0.01 mm.

13. The composition of claim 1, wherein the composition is a formulation for oral administration, administration by nasogastric tube, or administration by colonoscopy.

14. The composition of claim 1, wherein the composition is formulated for oral administration.

15. The composition of claim 1, wherein extract of human feces is from a donor who has not been exposed to antibiotics for at three months prior to sample collection.

16. The composition of claim 1, wherein the composition is lyophilized.

17. The composition of claim 16, wherein the composition wherein the composition has a water content of less than 5%.

18. The composition of claim 17, wherein the composition has a water content of less than 4%, 3%, 2%, 1% or 0.5%.

19. The composition of claim 1, wherein the composition is frozen.

20. The composition of claim 1, wherein the composition is a solid.

21. The composition of claim 1, wherein the composition has been frozen or lyophilized for at least 1 week, 2 weeks, 3 weeks, one month, 2 months, 3 months or 6 months.

22. The composition of claim 1, wherein the composition is in the form of a tablet, a troche, or a capsule.

23. The composition of claim 22, wherein the capsule comprises an acid-resistant enteric coating.

24. The composition of claim 23, wherein the coating comprises hypromellose (HPMC) or hypromellose phthalate (HPMCP).

25. The composition of claim 24, wherein the coating comprises a mixture of HPMC and HPMCP.

26. The composition of claim 23, wherein the capsule is formulated to release its contents in the small intestines.

27. The composition of claim 23, wherein the capsule has a volume of between 0.2 and 2 mls.

28. The composition of claim 1, wherein the composition is capable of being re-formulated for final delivery as comprising a liquid, a suspension, a gel, a geltab, a semisolid, a tablet, a sachet, a lozenge, a capsule, or as an enteral formulation.

29. The composition of claim 1, wherein the composition is formulated for multiple administrations.

30. The composition of claim 1, wherein the composition further comprises a saline, a defoaming agent, a surfactant agent, a lubricant, an acid neutralizer, a marker, a cell marker, a drug, an antibiotic, a contrast agent, a dispersal agent, a buffer or a buffering agent, a sweetening agent, a debittering agent, a flavoring agent, a pH stabilizer, an acidifying agent, a preservative, a desweetening agent, coloring agent, at least one vitamin, mineral supplement, a dietary supplement, a prebiotic nutrient or any combination thereof.

31. The composition of claim 1, wherein the composition comprises at least 1×10¹⁰, 1×10¹², 1.5×10¹², 2×10¹², or 2.5×10¹² bacteria.

32. An enteric-coated capsule comprising a lyophilized extract of human feces comprising (i) at least 1×10¹⁰ viable fecal microorganisms; (ii) a sugar; and (iii) a salt, wherein the enteric-coated capsule is formulated to release its contents in the small intestines upon oral administration to a human subject.

33. The capsule of claim 32, wherein the sugar comprises mannitol.

34. The capsule of claim 32, wherein the salt comprises sodium chloride.

35. The capsule of claim 32, wherein prior to lyophilization human feces are:
(i) blended with a salt solution and a sugar;
(ii) filtered to remove all solids larger than 0.5 mm; or
(iii) frozen for at least one hour.

36. The capsule of claim 35, wherein prior to lyophilization human feces are:
(i) blended with a salt solution and a sugar;
(ii) filtered to remove all solids larger than 0.5 mm; and
(iii) frozen for at least one hour.

37. The capsule of claim 35, wherein prior to lyophilization human feces are blended with a saline solution at a volume ratio of from 1:2 to 1:10 (feces to saline) and mannitol to final mannitol concentration of 0.5 to 5%.

38. The capsule of claim 35, wherein prior to lyophilization human feces are blended with a saline solution at a volume ratio of 1:5 (feces to saline) and mannitol to final mannitol concentration of 2%.
38.1. The capsule of claim 32, wherein the salt is present at about 0.5% to 1%.
38.2. The capsule of claim 38.1, wherein the salt is sodium chloride.
38.3. The capsule of claim 32, wherein the sugar is present at about 1% to 5%.
38.4. The capsule of claim 38.3, wherein the sugar is mannitol.

39. The capsule of claim 35, wherein the enteric coating comprises a mixture of HPMC and HPMCP.

40. The composition of any one of claims 1-39, wherein the composition is for therapeutic use to treat a patient having a disease characterized by dysfunctional microbiota.

41. The composition of claim 40, wherein the disease is selected from the group consisting of a Clostridium difficile colitis, a metabolic syndrome, obesity, asthma, eczema, an eosinophilic disorder of the gastrointestinal tract, inflammatory bowel disease, irritable bowel syndrome, ulcerative colitis, Crohn's disease, enterohemorrhagic colitis, chronic diarrhea, chronic constipation, an eating disorder, malnutrition, rheumatoid arthritis, systemic lupus erythematosus, multiple sclerosis, fibromyalgia, chronic fatigue syndrome, nonalcoholic fatty liver disease, and a neurodegenerative disorder.

42. The composition of claim 41, wherein the neurodegenerative disorder is Parkinson's disease.

43. The composition of claim 40, wherein the disease is a Clostridium difficile infection.

44. The composition of claim 43, wherein the Clostridium difficile infection is selected from an acute Clostridium difficile colitis, a relapsing Clostridium difficile colitis, and a severe Clostridium difficile colitis.

45. The composition of any one of claim 1-39, wherein the composition is for therapeutic use to replace a patient's microbiota.

46. The composition of claim 46, wherein some, most, or substantially all of the patient's colon, gut or intestinal microbiota are removed prior to the administering of the composition.

47. The composition of claim 46, wherein a single dosage of the composition comprises between 1×10¹⁰ and 5×10¹⁰ cells.

48. A method of preparing a composition comprising an extract of human feces comprising fecal microbiota, the method comprising: blending a fecal sample from a fecal donor with a diluent, filtering the blended fecal sample, and adding at least one sugar to the blended fecal sample.

49. A method of preparing a composition comprising an extract of human feces comprising fecal microbiota, the method comprising: blending a fecal sample from a fecal donor with a diluent and at least one sugar and filtering the blended fecal sample.

50. The method of claim 48 or 49, wherein the diluent is NaCl.

51. The method of claim 50, wherein the NaCl is further defined as 0.8% to 0.9% NaCl.

52. The method of claim 48 or 49, wherein the blending is performed at a 1:2 to 1:10 dilution (fecal sample to diluent).

53. The method of claim 48 or 49, wherein the blending is performed at a 1:4 to 1:6 dilution (fecal sample to diluent).

54. The method of claim 48 or 49, wherein the diluent does not comprise an antibacterial preservative.

55. The method of claim 48 or 49, wherein the blending is performed using a paddle blender.

56. The method of claim 48 or 49, wherein filtering comprises passing the fecal sample through a sieve and/or sedimenting solids from the fecal sample by centrifugation.

57. The method of claim 56, wherein the sieve is further defined as sterile gauze.

58. The method of claim 56, wherein the sieve comprises a sieve size of no greater than 2.0 mm, 1.0 mm, 0.5 mm, 0.25 mm, 0.212 mm, 0.180 mm, 0.150 mm, 0.125 mm, 0.106 mm, 0.090 mm, 0.075 mm, 0.063 mm, 0.053 mm, 0.045 mm, 0.038 mm, 0.032 mm, 0.025 mm, 0.020 mm, or 0.01 mm.

59. The method of claim 57, wherein the sterile gauze comprises 3, 4, 5, 6, or 7 layers.

60. The method of claim 57, wherein the sterile gauze comprises 5 layers.

61. The method of claim 57 or claim 60, wherein the sterile gauze is moistened with saline.

62. The method of claim 48 or 49, wherein filtering is performed more than once.

63. The method of claim 48 or 49, further comprising centrifuging a filtrate from the filtering step.

64. The method of claim 48 or 49, wherein the at least one sugar is mannitol.

65. The method of claim 48 or 49, wherein the at least one sugar is present at a concentration of 0.5% to 5% (vol/vol).

66. The method of claim 48 or 49, wherein the at least one sugar is present at a concentration of 1% to 3% (vol/vol).

67. The method of claim 48 or 49, wherein the at least one sugar is present at a concentration of at least 1%, 2%, 3%, 4%, 5%, 6%, 7%, 8%, 9%, or 10% (vol/vol).

68. The method of claim 48 or 49, further comprising freezing or lyophilizing the composition.

69. The method of claim 68, further comprising reconstituting the composition with an aqueous solution.

70. The method of claim 48 or 49, further comprising freezing the composition for at least 30 minutes, 1 hour, 2 hours, 3 hours or 4 hours.

71. The method of claim 70, wherein the sample is frozen for no more than 1 month, 1 week, 3 days or one day.

72. The method of claim 70, further comprising lyophilizing the frozen composition.

73. The method of claim 72, further comprising formulating the lyophilized composition in enteric capsules.

74. The method of claim 73, further comprising storing the enteric capsules at between 10 and 1° C.

75. A method for replacing, supplementing, or modifying a subject's colon microbiota, the method comprising administering to the subject the composition of any one of claims 1-39.

76. The method of claim 75, further comprising reconstituting the composition with an aqueous solution.

77. The method of claim 75, further comprising removal of some, most, or substantially all of the subject's colon, gut or intestinal microbiota prior to administering the composition.

78. The method of claim 75, wherein the composition is administered more than once.

79. The method of claim 75, wherein the composition is administered daily, weekly, or monthly.

80. The method of claim 75, wherein the composition is administered for two, three, or four months.

81. The method of claim 75, wherein the composition is administered orally or by colonoscopy.

82. A method for treating a subject having a disease characterized by dysfunctional microbiota, the method comprising administering to the subject in need thereof an effective amount of the composition of any one of claims 1-39.

83. The method of claim 82, further comprising removal of some, most, or substantially all of the subject's colon, gut or intestinal microbiota prior to administering the composition.

84. The method of claim 82, wherein the disease is selected from the group consisting of a Clostridium difficile colitis, a metabolic syndrome, obesity, asthma, eczema, an eosinophilic disorder of the gastrointestinal tract, inflammatory bowel disease, irritable bowel syndrome, ulcerative colitis, Crohn's disease, enterohemorrhagic colitis, chronic diarrhea, chronic constipation, an eating disorder, malnutrition, rheumatoid arthritis, systemic lupus erythematosus, multiple sclerosis, fibromyalgia, chronic fatigue syndrome, nonalcoholic fatty liver disease, and a neurodegenerative disorder.

85. The method of claim 84, wherein the neurodegenerative disorder is Parkinson's disease.

86. The method of claim 84, wherein the Clostridium difficile colitis is recurrent.

87. The method of claim 82, wherein the composition has been frozen or lyophilized for at least 1 week, 2 weeks, 3 weeks, one month, 2 months, 3 months or 6 months prior to the administration.

88. The method of claim 82, wherein the composition is administered more than once.

89. The method of claim 82, wherein the composition is administered daily, weekly, or monthly.

90. The method of claim 82, wherein the composition is administered for two, three, or four months.

91. The method of claim 82, wherein the composition is administered orally or by colonoscopy.

92. The method of claim 82, further comprising evaluating the subject by microbiome sequencing.

93. The method of claim 92, wherein administering the composition results in increased microbial diversity in the subject.

94. The method of claim 92, wherein administering the composition results in a decrease in the proportion of Proteobacteria in the subject's microbiota.

95. The method of claim 92, wherein administering the composition results in increase in the proportion of Firmicutes, Actinobacteria, Bacteroidetes and/or Verrucomicrobia in the subject's microbiota.
